(19) United States
(12) Patent Application Publication
(10) Pub. No.: US 2022/0113314 A1
(43) Pub. Date: Apr. 14, 2022

(54) METHOD OF AMPLIFYING FLUORESCENT SIGNAL THROUGH CYCLIC STAINING OF TARGET MOLECULES VIA FLUOROPHORE-CONJUGATED COMPLEMENTARY ANTIBODIES

(71) Applicant: Korea Advanced Institute Of Science And Technology, Daejeon (KR)

(72) Inventors: Jae-Byum CHANG, Daejeon (KR); Yehlin CHO, Daejeon (KR); Haemin YEON, Daejeon (KR); Junyoung SEO, Daejeon (KR); Yeonbo SIM, Daejeon (KR)

(21) Appl. No.: 17/495,133

(22) Filed: Oct. 6, 2021

(30) Foreign Application Priority Data
Oct. 8, 2020 (KR) 10-2020-0129915
Feb. 23, 2021 (KR) 10-2021-0024191
Jul. 20, 2021 (KR) 10-2021-0094785

(51) Int. Cl. G01N 33/58; G01N 33/53; G01N 21/64

(52) U.S. Cl. CPC G01N 33/582; G01N 33/5306; G01N 2021/6439; G01N 21/6428; G01N 21/6458

(57) ABSTRACT

Various embodiments can provide a method of amplifying a fluorescence signal through the cyclic staining of complementary antibodies conjugated with fluorophores. According to various embodiments, the method of amplifying a fluorescence signal may be configured to prepare a primary antibody and antibody pairs with respect to each target protein, bind the primary antibody to the target protein, and bind the antibody pairs to the primary antibody. The multicolor fluorescence signals may be amplified and fabricated by forming fluorescence signals in a way to prepare different antibodies for which cross reactions with different types of target proteins, respectively, have been removed using an agarose gel and to bind different antibody pairs according to combinations of different antibodies to different primary antibodies bound to target proteins, respectively.

### CROSS-REFERENCES TO RELATED APPLICATIONS

This application is based on and claims priority under 35 U.S.C. 119 to Korean Patent Application Nos. 10-2020-0129915 filed on Oct. 8, 2020, 10-2021-0024191 filed on Feb. 23, 2021, and 10-2021-0094785 filed on Jul. 20, 2021 in the Korean intellectual property office, the disclosures of which are herein incorporated by reference in their entireties.

### TECHNICAL FIELD

Various embodiments relate to a method of amplifying a fluorescence signal through the cyclic staining of target molecules via fluorophore-conjugated complementary antibodies. Furthermore, various embodiments relate to a method of amplifying multicolor fluorescence signals through the removal of a cross reaction between antibodies using an agarose gel.

### BACKGROUND OF THE INVENTION

Bioimaging is a noninvasive process of visualizing biological activity and distribution. Spatiotemporal distribution of the biomarkers enables the analysis of clinical specimens and tissue slices, thus helping to develop a therapy technology. As described above, the bioimaging technology has been developed as an innovative method for understanding a system within a living body by effectively visualizing a role and distribution of many proteins within a tissue. Furthermore, bioimaging may be applied as a biosensor for measuring bio markers within cells and tissue slices. Through the imaging technology capable of measuring a change in real time, a spatiotemporal distribution and function in a cell level can be understood. Furthermore, the imaging technology could be useful as a signal amplification technique in various fields, ranging from cell biology and neuroscience to medical research and medicine development. Bio imaging enables various drug responses associated with a disease to be visually identified, and can spatiotemporally provide a corresponding change. Bio imaging has been in the spotlight as an important tool for determining drug candidate substances in new drug development research and clinical fields and confirming a treatment effect, and utilization thereof is increased.

Recently developed tissue transparency technologies have expanded, to three-dimensional (3-D) large-volume imaging, Such tissue transparency schemes can minimize damage to a tissue, and enable cellular structures and a connection network to be checked. Accordingly, comprehensive mapping of a cell and a tissue in a molecular level will greatly improve a next-generation diagnosis and analysis system.

Despite such advantages, the 3-D high-volume imaging is very limited in that a high scan time is required. For example, large-volume immunofluorescence imaging can take anywhere from a few hours to a few days to complete, as it requires more than one million image acquisitions. This may be a fatal disadvantage in a modern medical system that requires large imaging data. However, such a problem may be solved by the amplification of a fluorescence signal of a sample. As the fluorescent intensity of a sample is increased, an exposure time necessary to obtain an image of the same fluorescent intensity is reduced. A reduction in the length of each image acquisition time through signal amplification could significantly reduce the total imaging time. Accordingly, a fluorescence signal amplification technology will play an important role in a next-generation bio imaging field that requires many image data.

A bioimaging technology that helps the diagnosis of a disease by measuring a bio marker within a living body requires high resolution. Furthermore, to find out an overall spatiotemporal distribution of and relation between multiple biomarkers by simultaneously imaging the multiple biomarkers involved with each other rather than the imaging of a single biomarker is a factor, that is, a key, in the bioimaging technology. Accordingly, technologies for signal amplification and simultaneous multicolor imaging are factors that must be satisfied for ultra sensitivity bio imaging and sensors.

Various fluorescence signal amplification technologies for obtaining high imaging throughput have been developed so far. However, many technologies have several limitations, such as a low signal to noise ratio, limited multicolor imaging, low spatial resolution, complexity and low accessibility.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Various embodiments provide a method of amplifying a fluorescence signal through the cyclic staining of target molecules via fluorophore-conjugated complementary antibodies.

Various embodiments provide a method of amplifying multicolor fluorescence signals through the removal of a cross reaction between orthogonal antibodies using an agarose gel.

Various embodiments provide a method of amplifying a fluorescence signal. The method of amplifying a fluorescence signal may include preparing a primary antibody and antibody pairs with respect to each target protein, binding the primary antibody to the target protein, and binding the antibody pairs to the primary antibody.

Various embodiments provide a method of amplifying multicolor fluorescence signals including multiple fluorescence signals amplified with respect to different types of target proteins, respectively. The method may include preparing different antibodies for each of which a cross reaction has been removed and amplifying the fluorescence signals by binding different antibody pairs according to combinations of different antibodies to different primary antibodies bound to the respective target proteins, respectively. Preparing the different antibodies includes removing the cross reactions with multiple orthogonal antibodies with respect to each of the different antibodies by using an agarose gel.

According to various embodiments, a fluorescence signal can be amplified. That is, various embodiments can simply implement an amplified fluorescence signal through an already commercialized antibody and a fluorescent molecule. This enables a fluorescence signal to be amplified and imaged through a conventional fluorescence microscope without additional equipment. Furthermore, according to various embodiments, multiple biomarkers labeling with multiple fluorophore-conjugated complementary can be amplified. Furthermore, an additional problem which may increase a background signal within a tissue can be limited because the purified complementary antibody pairs are orthogonal with biotin within a pathology tissue in the imaging of the pathology tissue.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A and 1B are diagrams illustrating methods for direct and indirect immunofluorescence.

FIGS. 2A and 2B are diagrams illustrating the amplification of a single fluorescence signal according to first embodiments.

FIGS. 3A and 3B are diagrams illustrating the amplification of a single fluorescence signal according to second embodiments.

FIG. 4 is a diagram illustrating a method of amplifying a single fluorescence signal according to various embodiments.

FIGS. 5A, 5B, 5C, 5D and 5E are diagrams for describing a method of amplifying a single fluorescence signal according to the first embodiments.

FIGS. 6A, 6B, 6C, 6D and 6E are diagrams for describing a method of amplifying a single fluorescence signal according to the second embodiments.

FIGS. 7A, 7B and 7C are diagrams for describing a degree of signal amplification of a single fluorescence signal according to various embodiments.

FIG. 8 is a diagram illustrating the amplification of multicolor fluorescence signals according to various embodiments.

FIG. 9 is a diagram for describing a common method of purifying antibodies using an agarose gel in which an NHS group has been activated.

FIG. 10 is a diagram for describing a method of purifying antibodies using an agarose gel in which an NHS group has been activated according to a first embodiment.

FIG. 11 is a diagram for describing a method of purifying antibodies using an agarose gel in which an NHS group has been activated according to a second embodiment.

FIG. 12 is a diagram for describing a method of purifying antibodies using an agarose gel in which an NHS group has been activated according to a third embodiment.

FIG. 13 is a diagram illustrating a method of amplifying multicolor fluorescence signals according to various embodiments.

FIG. 14 is a diagram more specifically illustrating a method of preparing each of different secondary antibodies in FIG. 13.

FIG. 15 is a diagram for describing degree of signal amplification of multicolor fluorescence signals according to various embodiments.

### DETAILED DESCRIPTION

While illustrative embodiments have been illustrated and described, it will be appreciated that various changes can be made therein without departing from the spirit and scope of the invention.

Hereinafter, various embodiments of this document are described with reference to the accompanying drawings.

Various embodiments propose a fluorescence signal amplification via cyclic staining of target molecules (FRACTAL) technology. According to various embodiments, an image processing apparatus processes an image through an amplified fluorescence signal. Various embodiments amplify an immunofluorescence signal by alternately using complementary antibodies to which fluorophores are bound, and thus overcome the limited image processing throughput. Furthermore, various embodiments remove a cross reaction of orthogonal antibody pairs by using an agarose gel spin column in which an NHS group has been activated, and then amplify multiple immunofluorescence signals through the cyclic staining of complementary antibodies, so that multiple channels can be imaged.

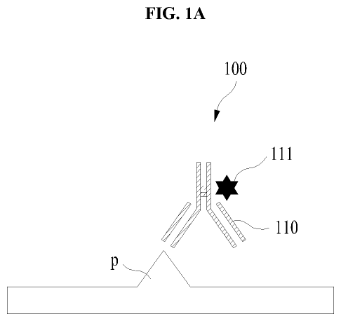

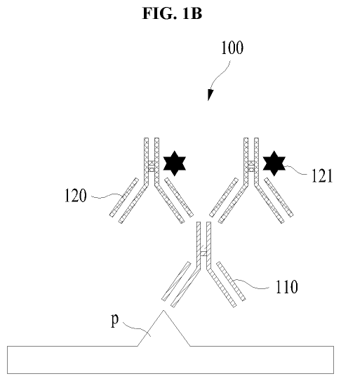

FIGS. 1A and 1B are diagrams illustrating methods for direct and indirect immunofluorescence.

Referring to FIGS. 1A and 1, a fluorescence signal 100 is provided without amplification. For example, the fluorescence signal 100 is labeled in a target protein “p” by a direct method. As illustrated in FIG. 1A, the fluorescence signal 100 consists of a primary antibody 110. The primary antibody 110 is conjugated with a fluorophore 111. For another example, the fluorescence signal 110 is labeled in the target protein “p” by an indirect method. As illustrated in FIG. 1B, the fluorescence signal 100 includes the primary antibody 110, and secondary antibodies 120 labeled to the primary antibody 110. The secondary antibodies 120 are conjugated with fluorophores 121, respectively. Such a fluorescence signal has a low image processing throughput.

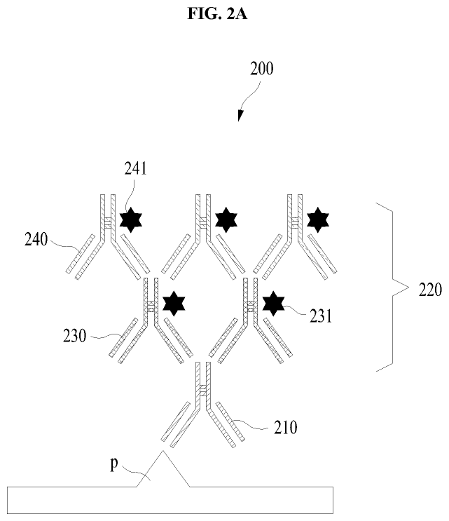

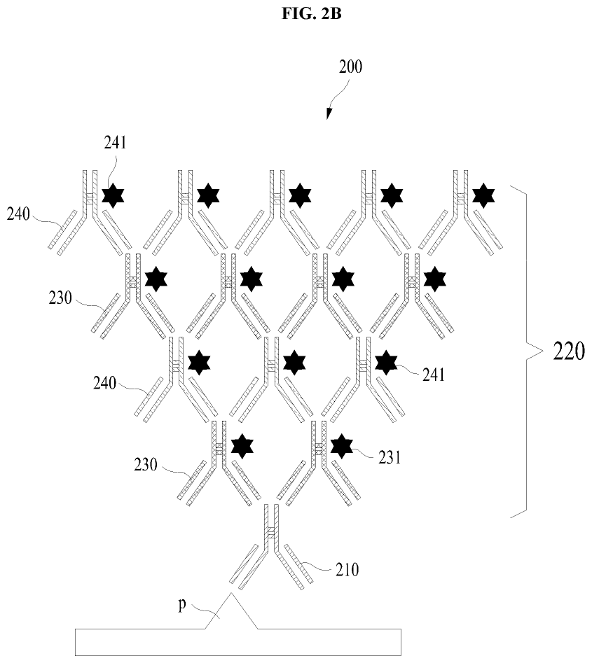

FIGS. 2A and 2B are diagrams illustrating the amplification of a single fluorescence signal 200 according to first embodiments.

Referring to FIGS. 2A and 2B, the amplified single fluorescence signal 200 according to the first embodiments is provided. The amplified single fluorescence signal 200 may include a primary antibody 210 and secondary antibody pairs 220. The primary antibody 210 may be labeled to a target protein “p”. The secondary antibody pairs 220 may be composed of first secondary antibodies 230 and second secondary antibodies 240. According to the first embodiments, both the first secondary antibodies 230 and the second secondary antibodies 240 may have been conjugated with fluorophores 231 and 241, respectively. In this case, the fluorophores 231 of the first secondary antibodies 230 and the fluorophores 241 of the second secondary antibodies 240 may be the same. In this case, the second secondary antibodies 240 may be labeled to the first secondary antibodies 230 within the secondary antibody pairs 220. The secondary antibody pairs 220 may be labeled while forming at least one layer from the primary antibody 210.

According to an embodiment, as illustrated in FIG. 2A, the secondary antibody pairs 220 may be bound while forming one layer from the primary antibody 210. In such a case, the secondary antibody pairs 220 may bound to the primary antibody 210 through the first secondary antibodies 230. The second secondary antibodies 240 may be labeled or bound to the first secondary antibodies 230. According to another embodiment, as illustrated in FIG. 2B, the secondary antibody pairs 220 may be labeled or bound to together as a periodical structure while forming multi-antibody layers from the primary antibody 210. In such a case, at least one of the secondary antibody pairs 220 may be bound to the primary antibody 210 through the first secondary antibodies 230. Furthermore, secondary antibody pairs 220 in a higher layer may be bound to second secondary antibodies 240 of the secondary antibody pairs 220 in a lower layer through first secondary antibodies 230.

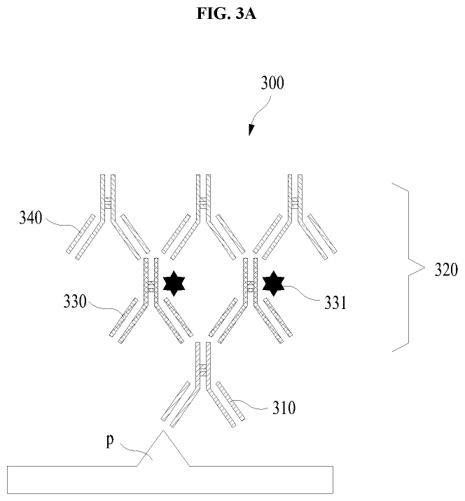

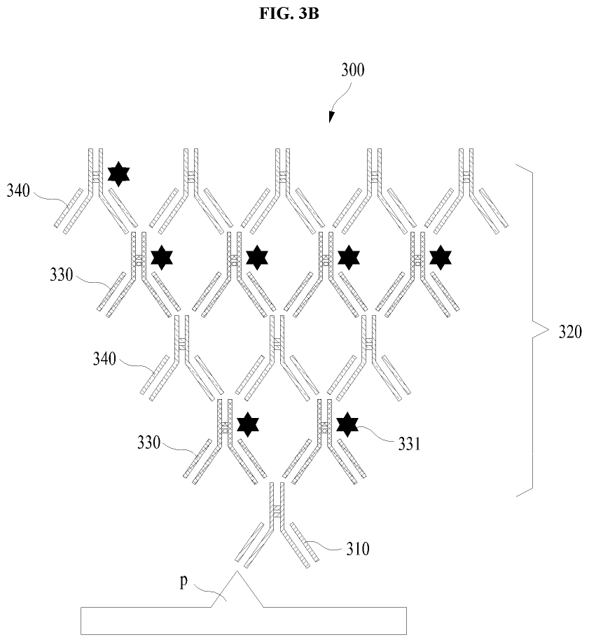

FIGS. 3A and 3B are diagrams illustrating the amplification of a single fluorescence signal 300 according to second embodiments.

Referring to FIGS. 3A and 3B, the amplified single fluorescence signal 300 according to the second embodiments is provided. The amplified single fluorescence signal 300 may include a primary antibody 310 and secondary antibody pairs 320. The primary antibody 310 may be bound to a target protein “p”. The secondary antibody pairs 320 may be composed of first secondary antibodies 330 and second secondary antibodies 340. According to the second embodiments, only the first secondary antibodies 330 may have been conjugated with fluorophores 331. In this case, the second secondary antibodies 340 may be bound to the first secondary antibodies 330 within the secondary antibody pairs 320. The secondary antibody pairs 320 may be bound while forming at least one layer from the primary antibody 310.

According to an embodiment, as illustrated in FIG. 3A, the secondary antibody pairs 320 may be bound while forming one layer from the primary antibody 310. In such a case, the secondary antibody pairs 320 may be bound to the primary antibody 310 through the first secondary antibodies 330. The second secondary antibodies 340 may be bound to the first secondary antibodies 330. According to another embodiment, as illustrated in FIG. 3B, the secondary antibody pairs 320 may be bonded together as a periodical structure while forming multi-antibody layers from the primary antibody 310. In such a case, at least any one of the secondary antibody pairs 320 may be bound to the primary antibody 310 through the first secondary antibodies 330. Furthermore, secondary antibody pairs 320 in a higher layer may be bound to second secondary antibodies 340 of the secondary antibody pairs 320 in a lower layer through first secondary antibodies 330.

According to various embodiments, a host of the first secondary antibodies 230, 330 may be different from a host of the primary antibodies 210, 310. A host of the second secondary antibodies 240, 340 may be the same as a host of the primary antibodies 210, 310. Furthermore, a target of the first secondary antibodies 230, 330 may be same as the host of the second secondary antibodies 240, 340. The host species of the first secondary antibodies 230, 330 may be identical so that of the target species of the second secondary antibodies 240, 340. In this case, the host species and the target species for the primary antibody 210, 310, first secondary antibodies 230, 330 and second secondary antibodies 240, 340 of the fluorescence signal 200, 300 may be determined based on the target protein “p” in which the fluorescence signal 200, 300 is stained.

For example, the primary antibody 210, 310, the first secondary antibodies 230, 330 and the second secondary antibodies 240, 340 of the fluorescence signal 200, 300 may be determined as host species and target species derived from various animals commercialized as in [Table 1] below. According to [Table 1] below, 64 types of secondary antibody pairs 220, 320 may be combined. For example, a host species of the primary antibody 210, 310 and the second secondary antibodies 240, 340 may be a rabbit, and a host species of the first secondary antibodies 230, 330 may be a donkey. In other words, the primary antibody 210, 310 may be rabbit primary antibodies, the first secondary antibodies 230, 330 may be donkey anti-rabbit secondary antibodies, and the second secondary antibodies 240, 340 may be rabbit anti-donkey secondary antibodies. For another example, host species of the primary antibody 210, 310 and the second secondary antibodies 240, 340 may be donkeys, and a host species of the first secondary antibodies 230, 330 may be a rabbit. In other words, the primary antibodies 210, 310 may be donkey primary antibodies, the first secondary antibodies 230, 330 may be rabbit anti-donkey secondary antibodies, and the second secondary antibodies 240, 340 may be donkey anti-rabbit secondary antibodies.

TABLE 1

| HOST | TARGET |
|---|---|
| rabbit | bovine |
| | chicken |
| | dog |
| | goat |
| | Syrian hamster |
| | horse |
| | human |
| | mouse |
| | rat |
| | sheep |
| | guinea pig |
| | Hamster |
| | camelid |
| | donkey |
| | cat |
| goat | bovine |
| | cat |
| | chicken |
| | guinea pig |
| | Armenian hamster |
| | Syrian hamster |
| goat | horse |
| | human |
| | mouse |
| | rabbit |
| | rat |
| | swine |
| | hamster |
| | dog |
| | camelid |
| | monkey |
| | pig |
| donkey | chicken |
| | goat |
| | guinea pig |
| | human |
| | rabbit |
| | rat |
| | sheep |
| | mouse |
| mouse | goat |
| | human |
| | rabbit |
| mouse | rat |
| | bovine |
| | monkey |
| | guinea pig |
| chicken | mouse |
| | human |
| | rat |
| | rabbit |
| | goat |
| alpaca | human |
| | mouse |
| | rabbit |
| sheep | mouse |
| | rat |
| | goat |
| | rabbit |
| bovine | goat |
| | human |
| | mouse |
| rat | mouse |
| horse | mouse |

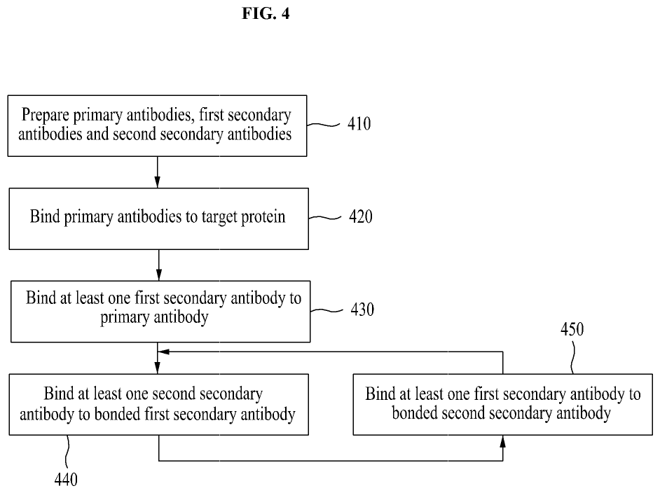

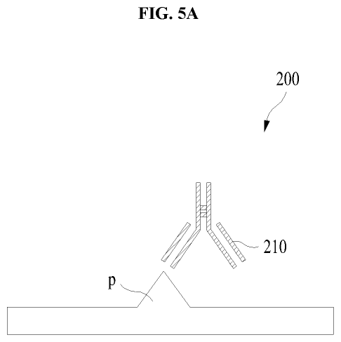

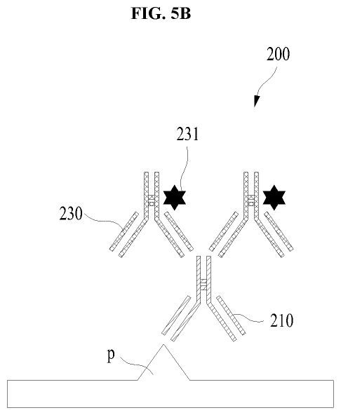

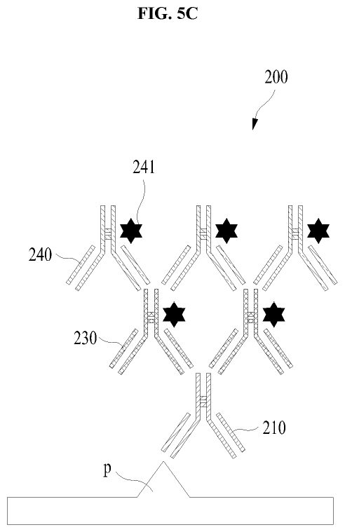

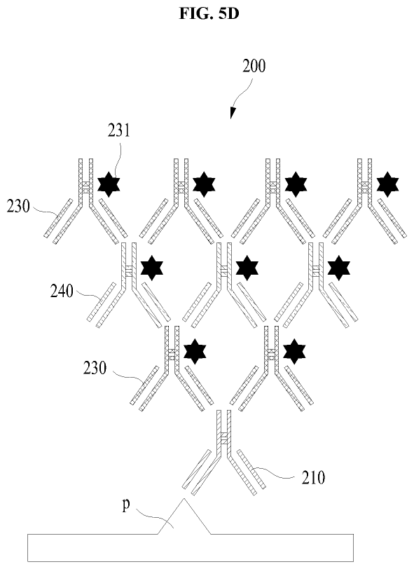

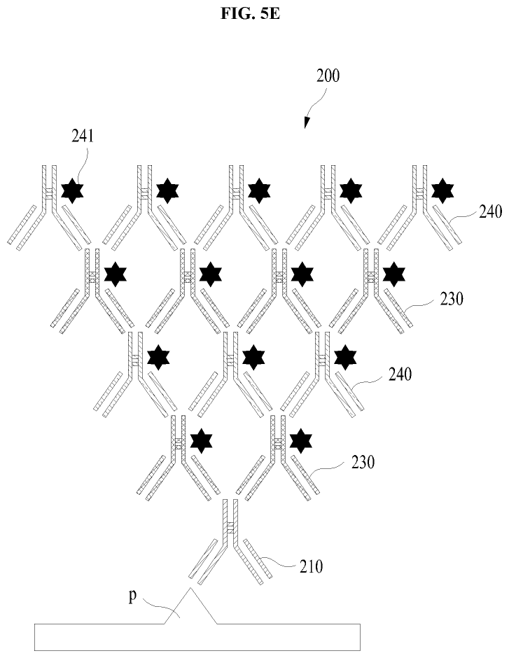

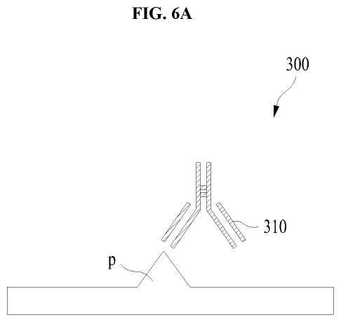

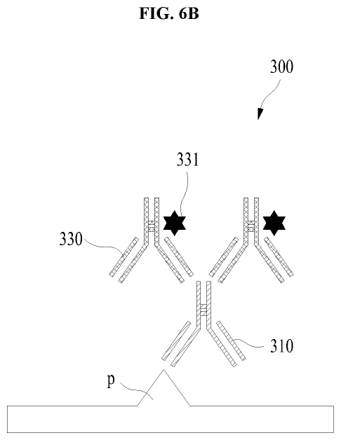

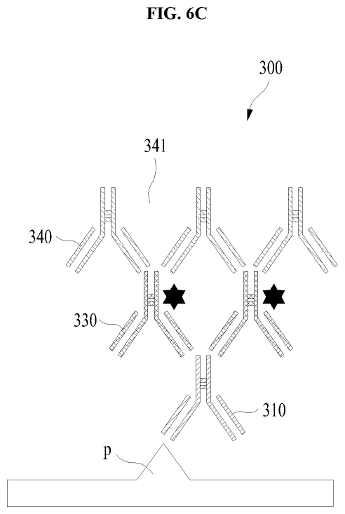

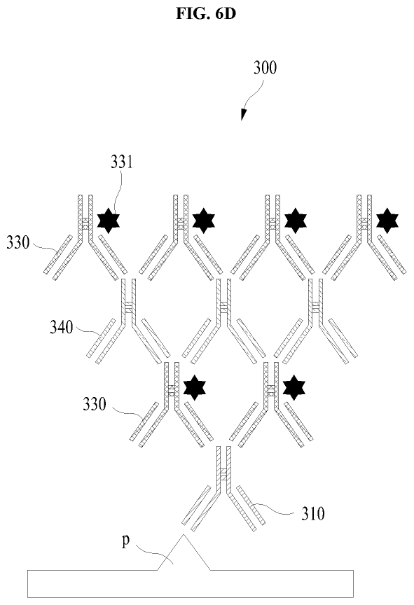

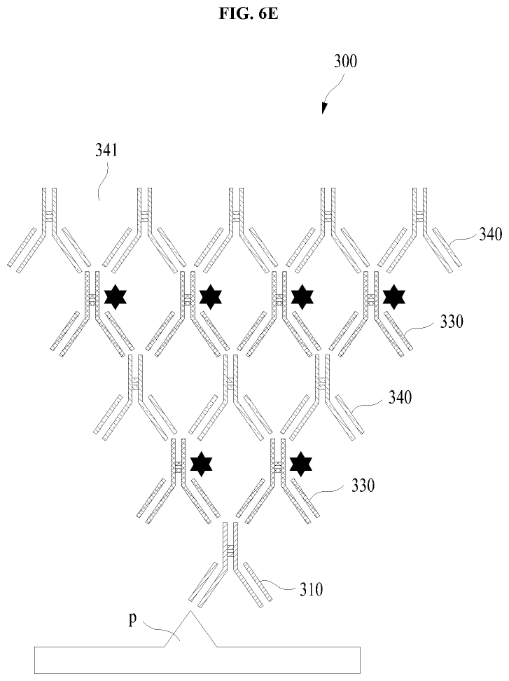

FIG. 4 is a diagram illustrating a method of amplifying the single fluorescence signal 200, 300 according to various embodiments. FIGS. 5A, 5B, 5C, 5D and 5E are diagrams for describing a method of amplifying the single fluorescence signal 200 according to the first embodiments. FIGS. 6A, 6B, 6C, 6D and 6E are diagrams for describing a method of amplifying the single fluorescence signal 300 according to the second embodiments.

Referring to FIG. 4, in step 410, the primary antibody 210, 310, the first secondary antibodies 230, 330 and the second secondary antibodies 240, 340 may be prepared. In this case, with respect to the target protein “p” to be stained, the primary antibody 210, 310, the first secondary antibodies 230, 330 and the second secondary antibodies 240, 340 may be prepared. According to the first embodiments, as illustrated in FIGS. 5A, 5B, 5C, 5D and 5E, both the first secondary antibodies 230 and the second secondary antibodies 240 may be conjugated with the fluorophores 231 and 241, respectively. In this case, the fluorophores 231 of the first secondary antibodies 230 and the fluorophores 241 of the second secondary antibodies 240 may be the same. According to the second embodiments, as illustrated in FIGS. 6A, 6B, 6C, 6D and 6E, only the first secondary antibodies 330 may be conjugated with the fluorophores 331.

According to various embodiments, a host species of the first secondary antibodies 230, 330 may be different from a host species of the primary antibody 210, 310. A host species of the second secondary antibodies 240, 340 may be the same as the host species of the primary antibody 210, 310. Furthermore, a target species of the first secondary antibodies 230, 330 may be the same as the host species of the second secondary antibodies 240, 340. The host species of the first secondary antibodies 230, 330 may be the same as a target species of the second secondary antibodies 240, 340. In this case, the host species and target species for the primary antibody 210, 310, first secondary antibodies 230, 330 and second secondary antibodies 240, 340 of the fluorescence signal 200, 300 may be determined based on the target protein “p” in which the single fluorescence signal 200, 300 is strained.

In step 420, the primary antibody 210, 310 may be bonded to the target protein “p”. As illustrated in FIG. 5A or 6A, the primary antibody 210, 310 may be bonded to the target protein “p”.

In steps 430 and 440, the first secondary antibodies 230, 330 and the second secondary antibodies 240, 340 may be bound while forming one layer from the primary antibody 210, 310. Specifically, in step 430, at least one first secondary antibody 230, 330 may be bonded to the primary antibody 210, 310. As illustrated in FIG. 5B or 6B, at least one first secondary antibody 230, 330 may be bonded to the primary antibody 210, 310. In this case, the first secondary antibodies 230, 330 may have been conjugated with the fluorophores 231, 331, respectively. Next, in step 440, at least one second secondary antibody 240, 340 may be bound to the bonded first secondary antibody 230, 330. As illustrated in FIG. 5C or 6C, at least one second secondary antibody 240, 340 may be bound to the first secondary antibody 230, 330 bound to the primary antibody 210, 310. According to the first embodiments, the second secondary antibodies 240 may have been conjugated with the fluorophores 241, respectively. In this case, the fluorophores 241 of the second secondary antibodies 240 may be the same as the fluorophores 231 of the first secondary antibodies 230. According to the second embodiments, the second secondary antibodies 340 may not have been conjugated with fluorophores.

Accordingly, the amplified single fluorescence signal 200, 300 according to an embodiment can be fabricated. That is, as illustrated in FIG. 5C or 6C, each of the amplified single fluorescence signals 200 and 300 in which each of the first secondary antibodies 230 and 330 and each of the second secondary antibodies 240 and 340 are bound together while forming one layer from each of the primary antibodies 210 and 310 can be fabricated.

Additionally, as steps 450 and 440 are repeated, the first secondary antibodies 230, 330 and the second secondary antibodies 240, 340 may be bound while forming multi-antibody layers from the primary antibody 210, 310. Specifically, in step 450, at least one first secondary antibody 230, 330 may be bound to the bound second secondary antibodies 240, 340. As illustrated in FIG. 5D or 6D, at least one first secondary antibody 230, 330 may be additionally bound to the second secondary antibodies 240, 340 bound to the first secondary antibodies 230, 330. Likewise, the first secondary antibodies 230, 330 may have been conjugated with the fluorophores 231, 331, respectively. Next, the process returns to step 440. In step 440, at least one second secondary antibody 240, 340 may be bound to the bound first secondary antibody 230, 330. As illustrated in FIG. 5E or 6E, at least one second secondary antibody 240, 340 may be additionally bound to the first secondary antibody 230, 330 bound to the second secondary antibodies 240, 340. According to the first embodiments, the second secondary antibodies 240 may have been conjugated with the fluorophores 241, respectively. According to the second embodiments, the second secondary antibodies 340 may not have been conjugated with fluorophores.

Accordingly, the amplified fluorescence signal 200, 300 according to another embodiment can be fabricated. That is, as illustrated in FIG. 5E or 6E, each of the amplified fluorescence signals 200 and 300 in which each of the first secondary antibodies 230 and 330 and each of the second secondary antibodies 240 and 340 are bound together while forming multi-antibody layers from each of the primary antibodies 210 and 310 can be fabricated. In this case, in the amplified single fluorescence signal 200, 300, the first secondary antibodies 230, 330 in a higher layer may be bound to the second secondary antibodies 240, 340 in a lower layer.

According to various embodiments, the amplified fluorescence signal 200, 300 can achieve a high image processing throughput. That is, the image processing throughput of the amplified single fluorescence signal 200, 300 may be much higher than that of the common fluorescence signal 100. In order to confirm such a comparison, the amplified single fluorescence signal 200 according to the first embodiments was fabricated so that the secondary antibody pairs 220 are bound while forming five layers from the primary antibody 210. As the results of a comparison between the corresponding single fluorescence signal 200 and the fluorescence signal 100, the corresponding single fluorescence signal 200 had increased by approximately 9.32-fold compared to the fluorescence signal 100. That is, compared to a case where the common fluorescence signal 100 was used, when the corresponding single fluorescence signal 200 was used, an imaging time necessary to obtain the same brightness could be reduced 9 times or more. For example, when a mouse brain slice (1×1×1 cm) for experiments is imaged, an imaging time according to the fluorescence signal 100 and an imaging time according to the corresponding single fluorescence signal 200 may be compared. In this case, the fluorescence signal 100 may require a total of an imaging time of 4 hours in conditions including an exposure time of 100 ms, magnification of 10, and a z-axis step size of 4 μm. In contrast, the corresponding fluorescence signal 200 can reduce an exposure time for obtaining an image having the same brightness to 10 ms. In this case, an imaging time for a brain slice can be reduced to about 20 to 30 minutes.

According to various embodiments, the amplified single fluorescence signal 200, 300 can be fabricated in a very simple way. For example, the amplified single fluorescence signal 200, 300 may be implemented even without a special chemical substance. In this case, the first secondary antibodies 230, 330 and the second secondary antibodies 240, 340 may form uniform layers through multiple bindings, so that the single fluorescence signal 200, 300 may be amplified. Various embodiments well operate even with an Expansion microscope (ExM), that is, the latest high-resolution imaging technology, and will be used as an innovative imaging technology having high-resolution and throughput.

According to the first embodiments, all of the first secondary antibodies 230 and the second secondary antibodies 240 may be conjugated with the fluorophores 231 and 241. In this case, a self-quenching of the fluorophores may occur between the fluorophores 231 and 241. According to the second embodiments, the first secondary antibodies 330 are conjugated with the fluorophores 331, but the second secondary antibodies 340 may not be conjugated with fluorophores. Accordingly, in the second embodiments, the degree of self-quenching may be decreased. As the degree of self-quenching is decreased, the amplified single fluorescence signal 300 according to the second embodiments may be amplified greater than the amplified single fluorescence signal 200 according to the first embodiments. For example, if the amplified single fluorescence signal 200 according to the first embodiments and the amplified fluorescence signal 300 according to the second embodiments have the same number of layers, the amplified single fluorescence signal 300 according to the second embodiments may be amplified about 1.5 times greater than the amplified fluorescence signal 200 according to the first embodiments. Furthermore, a manufacturing cost for the amplified single fluorescence signal 300 according to the second embodiments may be lower than a manufacturing cost for the amplified fluorescence signal 200 according to the first embodiments. Because the price of an unconjugated secondary antibody is approximately four times lower than that of a fluorophore-conjugated secondary antibody.

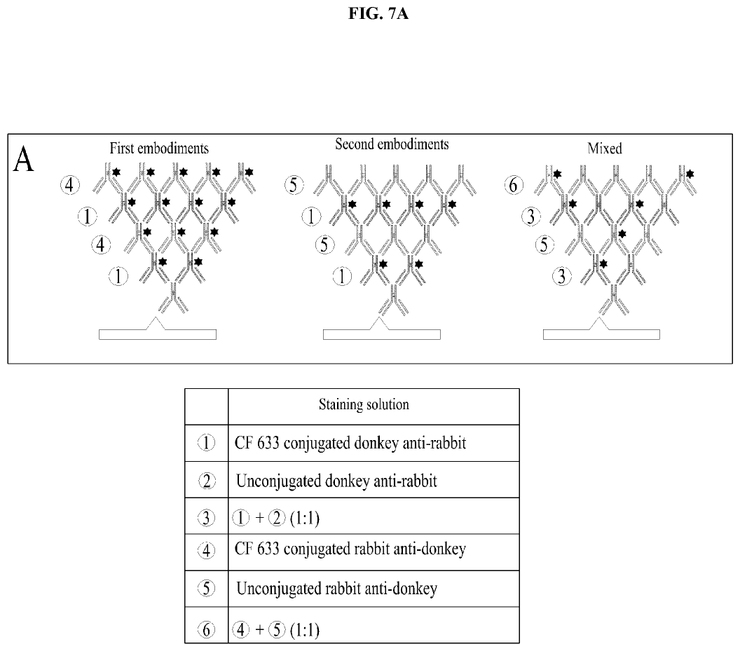

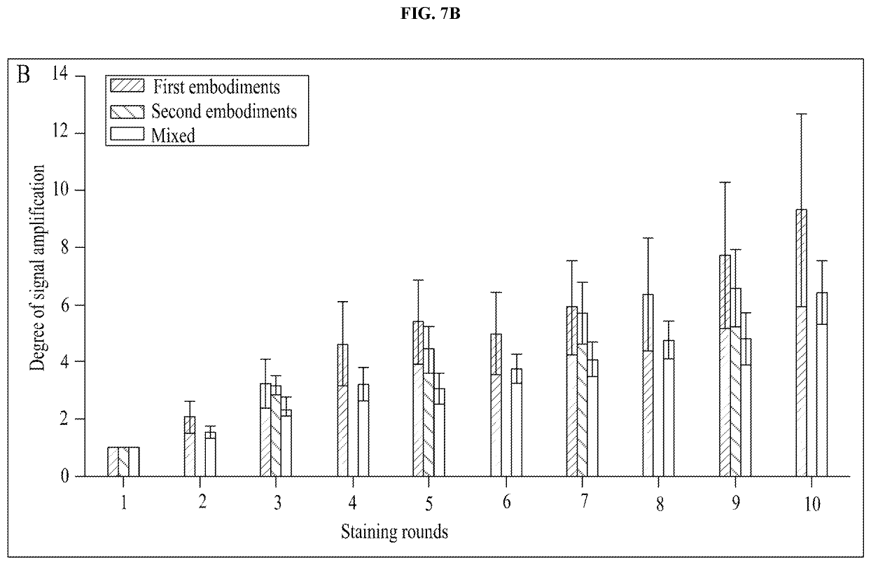

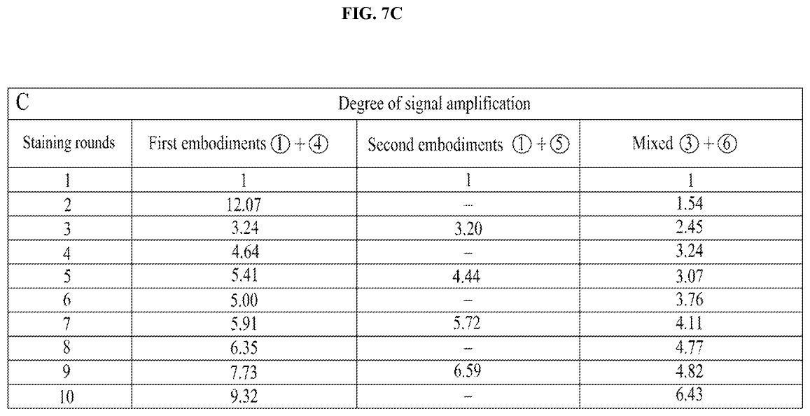

FIGS. 7A, 7B and 7C are diagrams for describing a degree of signal amplification of the single fluorescence signal 200, 300 according to various embodiments.

According to various embodiments, the single fluorescence signal 200, 300 may be amplified. Additionally, as illustrated in FIG. 7A, a single fluorescence signal according to third embodiments may be amplified as the first embodiments and the second embodiments are mixed. In such a case, only some of first secondary antibodies may be conjugated with fluorophores, and only some of second secondary antibodies may be conjugated with fluorophores. According to various embodiments, as illustrated in FIGS. 7B and 7C, as the number of layers of the secondary antibody pairs 220 is increased, that is, the number of dyes is increased, a degree of signal amplification of the single fluorescence signal 200, 300 may be increased.

A method of amplifying the fluorescence signal 200, 300 according to various embodiments may include a step (e.g., step 410) of preparing the primary antibody 210, 310, the first secondary antibodies 230, 330 conjugated with the fluorophores 231, 331, respectively, and the second secondary antibodies 240, 340 with respect to each of the target proteins “p”, a step (e.g., step 420) of binding the primary antibody 210, 310 to the target protein “p”, a step (e.g., step 430) of binding at least one first secondary antibody 230, 330 to the primary antibody 210, 310, and a step (e.g., step 440) of binding at least one second secondary antibody 240, 340 to the first secondary antibodies 230, 330.

According to various embodiments, the method of amplifying the fluorescence signal 200, 300 may further include a step (e.g., step 450) of binding at least one first secondary antibody 230, 330 to the second secondary antibodies 240, 340.

According to various embodiments, the step (i.e., step 450) of binding the first secondary antibodies 230, 330 and the step (i.e., step 440) of binding the second secondary antibodies 240, 340 may be repeatedly performed.

According to various embodiments, the second secondary antibodies 240, 340 may be conjugated with the fluorophores 241 and 341, respectively.

According to various embodiments, a host species of the first secondary antibodies 230, 330 may be different from a host species of the primary antibody 210, 310. A host species of the second secondary antibodies 240, 340 may be the same as the host species of the primary antibody 210, 310.

According to various embodiments, a target species of the first secondary antibodies 230, 330 may be the same as the host species of the second secondary antibodies 240, 340. The host species of the first secondary antibodies 230, 330 may be the same as a target species of the second secondary antibodies 240, 340.

According to various embodiments, the method of amplifying the fluorescence signal 200, 300 may be simultaneously performed on different types of target proteins “p”.

According to various embodiments, in the method of amplifying the fluorescence signal 200, 300, the primary antibody 210, 310, fluorophores, the first secondary antibodies 230, 330 and the second secondary antibodies 240, 340 may be different with respect to each of types.

According to various embodiments, the first secondary antibodies 230, 330 and the second secondary antibodies 240, 340 for one of types may be orthogonal to the first secondary antibodies 230, 330 and the second secondary antibodies 240, 340 for another of the types.

According to various embodiments, the first secondary antibodies 230, 330 and the second secondary antibodies 240, 340 for one of types may be purified and prepared with respect to the first secondary antibodies 230, 330 and the second secondary antibodies 240, 340 for another of the types.

According to various embodiments, if the target protein “p” is lamin B1, host species of the primary antibody 210, 310 and the second secondary antibodies 240, 340 may be rats, and a host species of the first secondary antibodies 230, 330 may be a mouse.

According to various embodiments, if the target protein “p” is vimentin, host species of the primary antibody 210, 310 and the second secondary antibodies 240, 340 may be chickens, and a host species of the first secondary antibodies 230, 330 may be a goat.

According to various embodiments, if the target protein “p” is tubulin, host species of the primary antibody 210, 310 and the second secondary antibodies 240, 340 may be rabbits, and a host species of the first secondary antibodies 230, 330 may be a donkey.

A method of amplifying the fluorescence signal 200, 300 according to various embodiments may include a step of forming the primary antibody 210, 310 bound to the target protein “p”, and a step of forming the secondary antibody pairs 220, 320 including the first secondary antibodies 230, 330 conjugated with the fluorophores 231, 331, respectively, and the second secondary antibodies 240, 340 bound to the first secondary antibodies 230, 330 with respect to the primary antibody 210, 310.

According to various embodiments, at least one of the secondary antibody pairs 220, 320 may be bound to the primary antibody 210, 310 through the first secondary antibodies 230, 330.

According to various embodiments, the secondary antibody pairs 220, 320 may be bound while forming multi-antibody layers from the primary antibody 210, 310.

According to various embodiments, a secondary antibody pair in a higher layer may be bound to the second secondary antibodies 240, 340 of a secondary antibody pair in a lower layer through the first secondary antibodies 230, 330.

According to various embodiments, the second secondary antibodies 240, 340 may be conjugated with the fluorophores 241 and 341, respectively.

According to various embodiments, a host species of the first secondary antibodies 230, 330 may be different from a host species of the primary antibody 210, 310. A host species of the second secondary antibodies 240, 340 may be the same as the host species of the primary antibody 210, 310.

According to various embodiments, a target species of the first secondary antibodies 230, 330 may be the same as the host species of the second secondary antibodies 240, 340. The host species of the first secondary antibodies 230, 330 may be the same as a target species of the second secondary antibodies 240, 340.

According to various embodiments, the primary antibody 210, 310, fluorophores, the first secondary antibodies 230, 330 and the second secondary antibodies 240, 340 may be differently determined depending on the type of target protein “p”.

According to various embodiments, if the target protein “p” is lamin B1, host species of the primary antibody 210, 310 and the second secondary antibodies 240, 340 may be rats, and a host species of the first secondary antibodies 230, 330 may be a mouse.

According to various embodiments, if the target protein “p” is vimentin, host species of the primary antibody 210, 310 and the second secondary antibodies 240, 340 may be chickens, and a host species of the first secondary antibodies 230, 330 may be a goat.

According to various embodiments, if the target protein “p” is tubulin, host species of the primary antibody 210, 310 and the second secondary antibodies 240, 340 may be rabbits, and a host species of the first secondary antibodies 230, 330 may be a donkey.

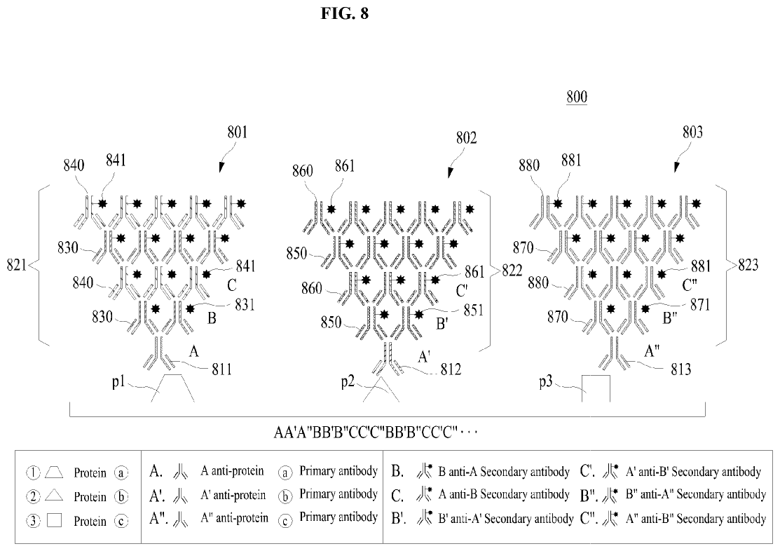

FIG. 8 is a diagram illustrating the amplification of a multi-fluorescence signal 800 according to various embodiments.

Referring to FIG. 8, the multi-fluorescence signal 800 according to various embodiments is provided. The multi-fluorescence signal 800 has been amplified by the amplification of a multi-fluorescence signal, that is, the multiplexed-FRACTAL technology, through the removal of cross reaction between orthogonal antibodies, and includes multiple fluorescence signals 801, 802, and 803 amplified with respect to different types of multiple target proteins p1, p2, and p3, respectively. In this case, each of the fluorescence signals 801, 802, and 803 is amplified with respect to each of the target proteins p1, p2, and p3 because a cross reaction of orthogonal antibody pairs between the fluorescence signals 801, 802, and 803 is removed.

Specifically, the fluorescence signals 801, 802, and 803 may include primary antibodies 811, 812, and 813 and secondary antibody pairs 821, 822, and 823, respectively. The primary antibodies 811, 812, and 813 may be bound to the corresponding target proteins p1, p2, and p3, respectively. The secondary antibody pairs 821, 822, and 823 may be composed of first secondary antibodies 830, 850, and 870 and second secondary antibodies 840, 860, and 880, respectively. According to an embodiment, all of the first secondary antibodies 830, 850, and 870 and the second secondary antibodies 840, 860, and 880 may have been conjugated with fluorophores 831, 851, and 871 and 841, 861, and 881, respectively. In this case, the fluorophores 831, 851, and 871 of the first secondary antibodies 830, 850, and 870 and the fluorophores 841, 861, and 881 of the second secondary antibodies 840, 860, and 880 may be the same, respectively. According to another embodiment, although not illustrated, only the first secondary antibodies 830, 850, and 870 may have been conjugated with the fluorophores 831, 851, and 871, respectively. In this case, the second secondary antibodies 840, 860, and 880 may be bound to the first secondary antibodies 830, 850, and 870 within the secondary antibody pairs 821, 822, and 823, respectively. Each of the secondary antibody pairs 821, 822, and 823 may be bound while forming at least one layer from each of the primary antibodies 811, 812, and 813. For example, each of the secondary antibody pairs 821, 822, and 823 may be bound as a periodical structure while forming multi-antibody layers from each of the primary antibodies 811, 812, and 813. In such a case, at least one of each of the secondary antibody pairs 821, 822, and 823 may be bound to each of the primary antibodies 811, 812, and 813 through each of the first secondary antibodies 830, 850, and 870. Furthermore, the secondary antibody pairs 821, 822, and 823 in a higher layer may be bound to the second secondary antibodies 840, 860, and 880 of secondary antibody pairs 821, 822, and 823 in a lower layer through the first secondary antibodies 830, 850, and 870, respectively.

According to various embodiments, the primary antibodies 811, 812, and 813, the first secondary antibodies 830, 850, and 870, the second secondary antibodies 840, 860, and 880 and the fluorophores 831 and 841, 851 and 861, and 871 and 881 may be differently determined with respect to the target proteins p1, p2, and p3. In particular, as a cross reaction of orthogonal antibody pairs, that is, the secondary antibody pairs 821, 822, and 823 between the fluorescence signals 801, 802, and 803, is removed, the secondary antibody pairs 821, 822, and 823 for one of types may be orthogonal to the secondary antibody pairs 821, 822, and 823 for the remainder of the types. Accordingly, the fluorescence signals 801, 802, and 803 are simultaneously stained with respect to different types of target proteins p1, p2, and p3, so that the multi-fluorescence signal 800 can be achieved.

According to various embodiments, a host species of each of the first secondary antibodies 830, 850, and 870 may be different from a host species of each of the primary antibodies 811, 812 and 813. A host species of each of the second secondary antibodies 840, 860, and 880 may be the same as a host species of each of the primary antibodies 811, 812, and 813. Furthermore, a target species of each of the first secondary antibodies 830, 850, and 870 may be the same as the host species of each of the second secondary antibodies 840, 860, and 880. The host species of each of the first secondary antibodies 830, 850, and 870 may be the same as a target species of each of the second secondary antibodies 840, 860, and 880. In this case, the host species and the target species for the primary antibodies 811, 812, and 813, the first secondary antibodies 830, 850, and 870 and the second secondary antibodies 840, 860, and 880 of the fluorescence signals 801, 802, and 803 may be determined based on the target proteins p1, p2, and p3 to be staimed, respectively.

For example, the primary antibodies 811, 812, and 813, first secondary antibodies 830, 850, and 870 and second secondary antibodies 840, 860, and 880 of the fluorescence signals 801, 802, and 803, respectively, may be determined as hosts and targets derived from various animals commercialized as in [Table 1]. According to [Table 1], the secondary antibody pairs 821, 822, and 823 may be combined using 64 types of the secondary antibodies 830 and 840, 850 and 860, and 870 and 880. For example, host species of the primary antibodies 811, 812, and 813 and the second secondary antibodies 840, 860, and 880 may be rabbits, and a host species of each of the first secondary antibodies 830, 850, and 870 may be a donkey. In other words, each of the primary antibodies 811, 812, and 813 may be a rabbit primary antibody, the first secondary antibodies 830, 850, and 870 may be donkey anti-rabbit secondary antibodies, and the second secondary antibodies 840, 860, and 880 may be rabbit anti-donkey secondary antibodies. For another example, host species of the primary antibodies 811, 812, and 813 and the second secondary antibodies 840, 860, and 880 may be donkeys, and host species of the first secondary antibodies 830, 850, and 870 may be rabbits. In other words, the primary antibodies 811, 812, and 813 may be donkey primary antibodies, the first secondary antibodies 830, 850, and 870 may be rabbit anti-donkey secondary antibodies, and the second secondary antibodies 840, 860, and 880 may be donkey anti-rabbit secondary antibodies.

According to various embodiments, a cross reaction of each of the secondary antibody pairs 821, 822, and 823 may be removed using an agarose gel. Specifically, after an antibody is fixed through an amide bond between an NHS group on a surface of the agarose gel and an amine group on a surface of each of the secondary antibody pairs 821, 822, and 823, a cross reaction may be removed through a process of purifying each of the secondary antibody pairs 821, 822, and 823. In this case, a cross reaction of each of the secondary antibody pairs 821, 822, and 823 for the remainder of types can be easily removed through a single purification process for each of the secondary antibody pairs 821, 822, and 823 for one of the types. For example, rabbit anti-donkey antibodies may be purified with respect to mouse anti-rat antibodies, rat anti-mouse antibodies, goat anti-chicken antibodies, and chicken anti-goat antibodies.

In the aforementioned description, the secondary antibody pairs 821, 822, and 823 have been described as being bound to the primary antibodies 811, 812, and 813, respectively, but the present disclosure is not limited thereto. Antibody pairs consisting of orthogonal antibodies whose cross reaction has been removed, other than the secondary antibody pairs 821, 822, and 823, may be bound to each of the primary antibodies 811, 812, and 813. In other words, the antibody pairs bound to each of the primary antibodies 811, 812, and 813 do not need to be essentially composed of each of the secondary antibodies 830 and 840, 850 and 860, and 870 and 880. For example, the antibody pairs bound to the primary antibody may be composed of two antibodies one of which modified with a protein molecule at Fc region and the other having the molecule as a target. Accordingly, limitations to the selection of an antibody for the amplification of the multi-fluorescence signal 800 may be reduced. This will be identically applied to the following description. Furthermore, not even antibody pairs but also molecule pairs that have selective and complementary affinity to each other can be used for cyclic staining.

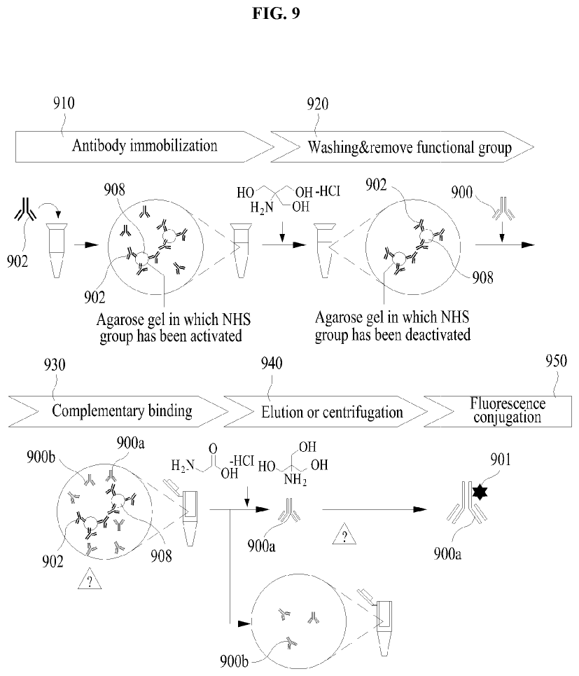

FIG. 9 is a diagram for describing a common method of purifying antibodies 900 using an agarose gel in which an NHS group has been activated.

Referring to FIG. 9, first, in step 910, the complementary antibodies 902 to the antibodies 900 to be purified may be put in an agarose gel 908 along with 1× phosphate buffer saline (PBS). In this case, an NHS group on a surface of the agarose gel 908 and an amine group of complementary antibodies 902 react with each other to form an amide bond. The complementary antibodies 902 may be connected to the agarose gel 908. Thereafter, in step 920, in order to remove the remaining NHS groups, the agarose gel 908 may be treated using a trisaminomethane solution of 1M. Next, in step 930, the antibodies 900 to be purified are put in the agarose gel 908 along with 1×PBS. Accordingly, the antibodies 900 may react with the complementary antibodies 902. In this case, antibodies 900a having high affinity among the antibodies 900 to be purified may be bound to the complementary antibodies 902. Next, in step 940, antibodies 900b not bound to the agarose gel 908 may be removed through centrifugation. Alternatively, in step 940, the antibodies 900a bound to the complementary antibodies 902 may be obtained through an elution process. Next, in step 950, the obtained antibodies 900a are conjugated with fluorophores 901, and may be used for staining of the target proteins.

However, the method of FIG. 9 may have two great problems. The first problem is that since the antibodies 900a having high affinity with the complementary antibodies 902 are only purified, a cross reaction between the antibodies 900a and their orthogonal antibodies is not removed. The second problem is that the purified antibodies 900a are not properly conjugated with the fluorophores 901 due to an unstable acidity condition in an elution process. A maximum yield in the process of conjugating the fluorophores 901 is obtained at acidity of 8.3, whereas the acidity of a glycine solution of 1M used in the elution process is about 2.5 to 3.0.

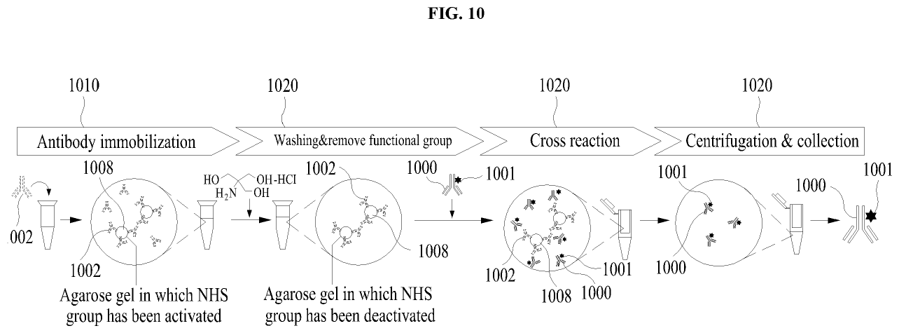

FIG. 10 is a diagram for describing a method of purifying antibodies 1000 using an agarose gel in which an NHS group has been activated according to a first embodiment. In this case, FIG. 10 is for describing a method of purifying against the one type of orthogonal antibody 1002, which solves the problems of the method.

Referring to FIG. 10, first, in step 1010, in order to solve the first problem, orthogonal antibodies 1002 to the antibodies 1000 to be purified may be connected to an agarose gel 1008. Specifically, the orthogonal antibodies 1002 may be put in the agarose gel 1008 along with 1×PBS. In this case, an NHS group on a surface of the agarose gel 1008 and an amine group of the orthogonal antibodies 1002 react with each other to form an amide bond. The orthogonal antibodies 1002 may be connected to the agarose gel 1008. Thereafter, in step 1020, in order to remove the remaining NHS groups, the agarose gel 1008 may be treated using a trisaminomethane solution of 1M.

Next, in step 1030, in order to solve the second problem, the antibodies 1000 to be purified may be previously conjugated with fluorophores 1001 and then put in the agarose gel 1008. Specifically, the antibodies 1000 conjugated with the fluorophores 1001 may be put in the agarose gel 1008 along with the 1× PBS. Accordingly, in step 1030, some of the antibodies 1000 to be purified are cross absorbed to the orthogonal antibodies 1002. Next, in step 1040, antibodies 1000 not cross absorbed to the orthogonal antibodies 1002, that is, the remaining antibodies 1000, may be obtained. Accordingly, a cross reaction between the remaining antibodies 1000 and the orthogonal antibodies 1002 may be removed. That is, the remaining antibodies 1000 may be composed of orthogonal secondary antibody pairs 821, 822, 823 different from the orthogonal antibodies 1002 without an elution process, and may be used as two types of the fluorescence signals 801, 802, 803.

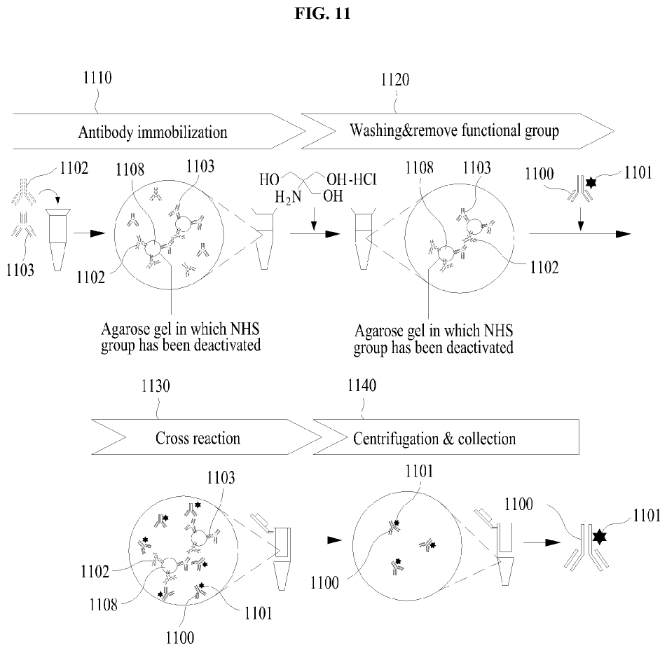

FIG. 11 is a diagram for describing a method of purifying antibodies 1100 using an agarose gel in which an NHS group has been activated according to a second embodiment. In this case, FIG. 11 is for describing a method of purifying two types of orthogonal antibodies 1102 and 1103.

Referring to FIG. 11, first, in step 1110, two types of antibodies 1102 and 1103 orthogonal to the antibodies 1100 to be purified and orthogonal to each other may be connected to the agarose gel 1108. Specifically, the orthogonal antibodies 1102 and 1103 may be put in the agarose gel 1108 along with 1×PBS. In this case, an NHS group on a surface of the agarose gel 1108 and an amine group of each of the orthogonal antibodies 1102 and 1103 react with each other to form an amide bond. The orthogonal antibodies 1102 and 1103 may be connected to the agarose gel 1108. Thereafter, in step 1120, in order to remove the remaining NHS groups, the agarose gel 1108 may be treated using a trisaminomethane solution of 1M.

Next, in step 1130, the antibodies 1100 to be purified are previously conjugated with fluorophores 1101 and then put in the agarose gel 1108. Specifically, the antibodies 1100 conjugated with the fluorophores 1101 may be put in the agarose gel 1108 along with the 1× PBS. Accordingly, in step 1130, some of the antibodies 1100 to be purified and cross absorbed to the orthogonal antibodies 1102 and 1103. Next, in step 1140, antibodies 1100 not bound to the orthogonal antibodies 1102 and 1103, that is, the remaining antibodies 1100, may be obtained. Accordingly, cross reactions between the remaining antibodies 1100 and the two types of orthogonal antibodies 1102 and 1103 can be simultaneously removed. That is, the remaining antibodies 1100 are composed of the orthogonal secondary antibody pairs 821, 822, and 823 different the orthogonal antibodies 1102 and 1103 without an elution process, and may be used as three types of fluorescence signals 801, 802, and 803.

However, if the secondary antibody pairs 821, 822, and 823 are constructed using the antibodies 1100 obtained by the method of FIG. 11, a cross reaction may occur between the second layer of the lowest layer bonded to each of the first secondary antibodies 830, 850, and 870 and the first layer of the higher layer. Specifically, each of the first secondary antibodies in the third layer 830, 850, and 870 may generate a cross reaction with respect to each of the second secondary antibodies 840, 860, and 880 in the second layer.

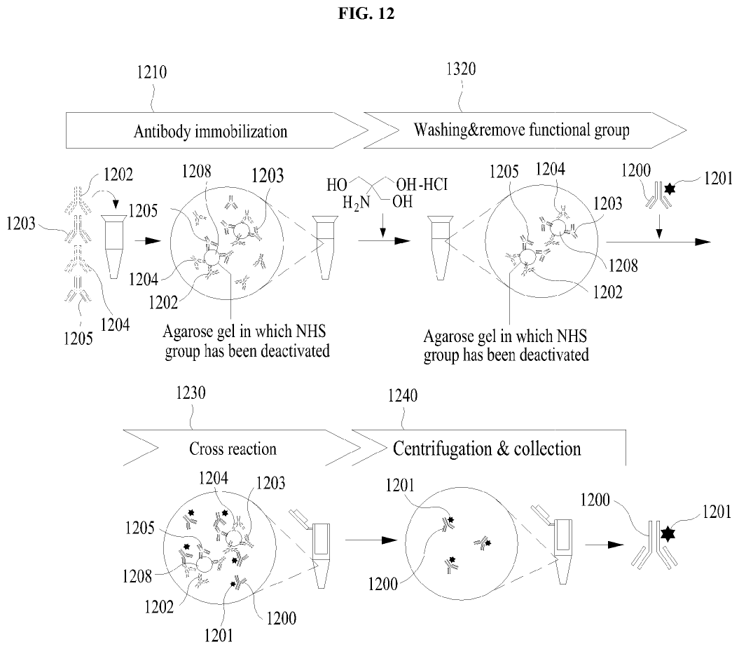

FIG. 12 is a diagram for describing a method of purifying antibodies 1200 using an agarose gel in which an NHS group has been activated according to a third embodiment. In this case, FIG. 12 is for solving the problems of the purification method of FIG. 11, and describes a method of purifying against the all orthogonal antibodies, for example, four types of orthogonal antibodies 1202, 1203, 1204, and 1205.

Referring to FIG. 12, first, in step 1210, all of the antibodies 1202, 1203, 1204, and 1205 orthogonal to the antibodies 1200 to be purified and orthogonal to each other may be connected to the agarose gel 1208. Specifically, the orthogonal antibodies 1202, 1203, 1204, and 1205 may be put in the agarose gel 1208 along with 1×PBS. In this case, an NHS group on a surface of the agarose gel 1208 and an amine group of each of the orthogonal antibodies 1202, 1203, 1204, and 1205 react with each other to form an amide bond. The orthogonal antibodies 1202, 1203, 1204, and 1205 may be connected to the agarose gel 1208. Thereafter, in step 1220, in order to remove the remaining NHS groups, the agarose gel 1208 may be treated using a trisaminomethane solution of 1M.

Next, in step 1230, the antibodies 1200 to be purified may be previously conjugated with fluorophores 1201 and then put in the agarose gel 1208. Specifically, the antibodies 1200 conjugated with the fluorophores 1201 may be put in the agarose gel 1208 along with the 1× PBS. Accordingly, in step 1230, some of the antibodies 1200 to be purified cross absorbed to the orthogonal antibodies 1202, 1203, 1204 and 1205. Next, in step 1240, antibodies 1200 not cross absorbed to the orthogonal antibodies 1202, 1203, 1204, and 1205, that is, the remaining antibodies 1200, may be obtained. Accordingly, cross reactions between the remaining antibodies 1200 and all of the orthogonal antibodies 1202, 1203, 1204, and 1205 can be removed at once. That is, the remaining antibodies 1200 may be composed of the orthogonal secondary antibody pairs 821, 822, and 823 different from the orthogonal antibodies 1202, 1203, 1204, and 1205, and may be used as several types of fluorescence signals 801, 802, and 803. Accordingly, in each of the fluorescence signals 801, 802, and 803, a cross reaction in each of the secondary antibody pairs 821, 822, and 823 and a cross reaction between layers may be fully removed.

As described above, one antibody 1200 may be purified with respect to all of the orthogonal antibodies 1202, 1203, 1204, and 1205, for example, the four orthogonal antibodies 1202, 1203, 1204, and 1205. For example, a mouse anti-rat antibody may be purified with respect to a goat anti-chicken antibody, a chicken anti-goat antibody, a donkey anti-rabbit antibody, and a rabbit anti-donkey antibody. In this manner, multiple antibodies may be purified, and a cross reaction between the antibodies may be removed. Accordingly, the multiple orthogonal secondary antibody pairs 821, 822, and 823 are composed of combinations of multiple antibodies, so that the multiple fluorescence signals 801, 802, and 803 may be implemented. For example, six types of antibodies may be purified, so that the three types of secondary antibody pairs 821, 822, and 823 are constructed. Furthermore, the three fluorescence signals 801, 802, and 803 may be implemented. That is, in order to implement the multi-fluorescence signal 800 composed of n fluorescence signals 801, 802, and 803, all antibodies used may be purified against the (2n-2) antibodies at a time.

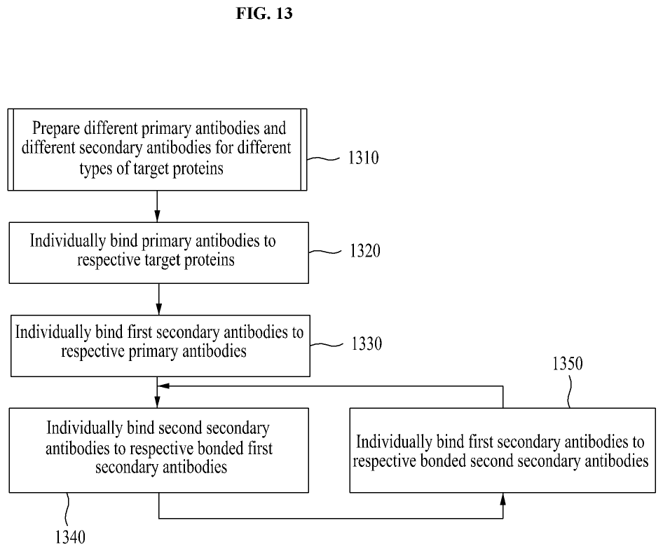

FIG. 13 is a diagram illustrating a method of amplifying the multi-fluorescence signal 800 according to various embodiments.

Referring to FIG. 13, in step 1310, for different types of the target proteins p1, p2, and p3, the different primary antibodies 811, 812 and 813, and the different secondary antibodies 830 and 840, 850 and 860, and 870 and 880 may be prepared, respectively. In this case, the primary antibodies 811, 812, and 813 and the secondary antibodies 830 and 840, 850 and 860, and 870 and 880 may be differently determined depending on the types of target proteins p1, p2, and p3 to be stained, respectively. In this case, the secondary antibodies 830 and 840, 850 and 860, and 870 and 880 may be prepared by being divided into the first secondary antibodies 830, 850, and 870 and the second secondary antibodies 840, 860, and 880. According to the first embodiments, all of the secondary antibodies 830 and 840, 850 and 860, and 870 and 880 may be conjugated with the fluorophores 831 and 841, 851 and 861, and 871 and 881, respectively. In this case, the fluorophores 831, 851, and 871 of the respective first secondary antibodies 830, 850, and 870 and the fluorophores 841, 861, and 881 of the respective second secondary antibodies 840, 860, and 880 may be the same. According to the second embodiments, although not illustrated, only the first secondary antibodies 830, 850, and 870 may be conjugated with the fluorophores 831, 851, and 871, respectively.

According to various embodiments, a host species of each of the first secondary antibodies 830, 850, and 870 may be different from a host species of each of the primary antibodies 811, 812 and 813. A host species of each of the second secondary antibodies 840, 860, and 880 may be the same as the host species of each of the primary antibodies 811, 812, and 813. Furthermore, a target species of each of the first secondary antibodies 830, 850, and 870 may be the same as the host species of each of the second secondary antibodies 840, 860, and 880. The host species of each of the first secondary antibodies 830, 850, and 870 may be the same as a target speciesof each of the second secondary antibodies 840, 860, and 880.

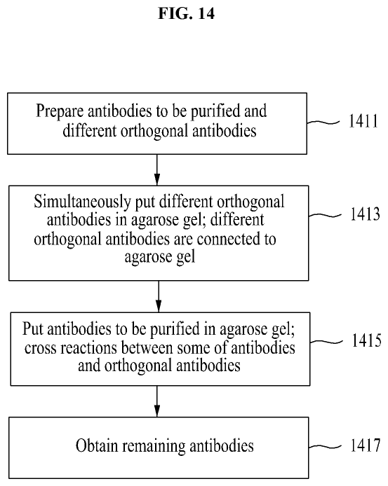

According to various embodiments, a cross reaction between each of the different secondary antibodies 830 and 840, 850 and 860, and 870 and 880 and multiple orthogonal antibodies may be removed using an agarose gel. In this case, as each of the different secondary antibodies 830 and 840, 850 and 860, and 870 and 880 is purified, a cross reaction with each of the different secondary antibodies 830 and 840, 850 and 860, and 870 and 880 may be removed. For example, each of antibodies whose host species and target species are a rat and a mouse and antibodies whose host species and target species are a mouse and a rat, antibodies whose host species and target species are a chicken and a goat and antibodies whose host species and target species are a goat and a chicken, and antibodies whose host species and target species are a rabbit and a donkey and antibodies whose host species and target species are a donkey and a rabbit may be orthogonal to each other. This will be more specifically described with reference to FIG. 14.

FIG. 14 is a diagram more specifically illustrating a method of preparing each of the different secondary antibodies 830 and 840, 850 and 860, and 870 and 880 in FIG. 13. In this case, FIG. 14 illustrates a method of preparing one of the different secondary antibodies 830 and 840, 850 and 860, and 870 and 880. The method may be applied to each of the different secondary antibodies 830 and 840, 850 and 860, and 870 and 880 in the same manner. Accordingly, all of the different secondary antibodies 830 and 840, 850 and 860, and 870 and 880 may be prepared.

Referring to FIG. 14, in step 1411, the antibodies 1200 to be purified and different orthogonal antibodies 1202, 1203, 1204, and 1205 orthogonal to the antibodies 1200 to be purified, respectively, may be prepared. In this case, the antibodies 1200 to be purified may be conjugated with the fluorophores 1201. Furthermore, the different orthogonal antibodies 1202, 1203, 1204, and 1205 may include all of orthogonal antibodies 1202, 1203, 1204, and 1205 orthogonal to the antibodies 1200 to be purified, respectively.

Next, in step 1413, the different orthogonal antibodies 1202, 1203, 1204, and 1205 may be simultaneously put in the agarose gel 1208. Specifically, the different orthogonal antibodies 1202, 1203, 1204, and 1205 may be put in the agarose gel 1208 along with 1×PBS. In this case, an NHS group on a surface of the agarose gel 1208 and an amine group of each of the orthogonal antibodies 1202, 1203, 1204, and 1205 react with each other to form an amide bond. Accordingly, the orthogonal antibodies 1202, 1203, 1204, and 1205 may be connected to the agarose gel 1208.

Next, in step 1415, the antibodies 1200 to be purified may be put in the agarose gel 1208. In this case, in order to remove the remaining NHS groups at the surface of the agarose gel 1208, the agarose gel 1208 is treated with a trisaminomethane solution of 1M. Thereafter, the antibodies 1200 to be purified may be put in the agarose gel 1208. Accordingly, some of the put antibodies 1200 may be cross absorbed to the different orthogonal antibodies 1202, 1203, 1204, and 1205.

Next, in step 1417, antibodies 1200 not bound to the different orthogonal antibodies 1202, 1203, 1204, and 1205, that is, the remainder of the put antibodies 1200, may be obtained through centrifugation. Accordingly, as the remaining antibodies 1200 are purified with respect to the different orthogonal antibodies 1202, 1203, 1204, and 1205, for example, four orthogonal antibodies 1202, 1203, 1204, and 1205, cross reactions with all of the orthogonal antibodies 1202, 1203, 1204, and 1205 can be simultaneously removed. For example, a mouse anti-rat antibody may be purified with respect to a goat anti-chicken antibody, a chicken anti-goat antibody, a donkey anti-rabbit antibody, and a rabbit anti-donkey antibody. Accordingly, the remaining antibodies 1200 may be obtained as one of the different secondary antibodies 830 and 840, 850 and 860, and 870 and 880. In this manner, as each of the different secondary antibodies 830 and 840, 850 and 860, and 870 and 880 are purified, a cross reaction with each of the different secondary antibodies 830 and 840, 850 and 860, and 870 and 880 may be removed.

Referring back to FIG. 13, in step 1320, the different primary antibodies 811, 812 and 813 may be individually bound to the target proteins p1, p2, and p3. Next, in steps 1330 and 1340, each of the different first secondary antibodies 830, 850, and 870 and each of the different second secondary antibodies 840, 860, and 880 may be bound together while forming one layer from each of the different primary antibodies 811, 812 and 813. Specifically, in step 1330, the different first secondary antibodies 830, 850, and 870 may be individually bound to the different primary antibodies 811, 812 and 813, respectively. Next, in step 1340, the different second secondary antibodies 840, 860, and 880 may be individually bound to the bound first secondary antibodies 830, 850, and 870, respectively.

Accordingly, the amplified multi-fluorescence signal 800 according to an embodiment can be fabricated. That is, the multiple amplified fluorescence signals 801, 802, and 803 in which each of the different first secondary antibodies 830, 850, and 870 and each of the different second secondary antibodies 840, 860, and 880 are bound together while forming one layer from each of the different primary antibodies 811, 812 and 813 can be simultaneously fabricated. Accordingly, the amplified multi-fluorescence signal 800 can be fabricated.

Additionally, as steps 1350 and 1340 are repeated, each of the different first secondary antibodies 830, 850, and 870 and each of the different second secondary antibodies 840, 860, and 880 may be bound together while forming multi-antibody layers from each of the different primary antibodies 811, 812 and 813. Specifically, in step 1350, the different first secondary antibodies 830, 850, and 870 may be individually and additionally bound to the bound second secondary antibodies 840, 860, and 880, respectively. Next, the process returns to step 1340. In step 1340, the different second secondary antibodies 840, 860, and 880 may be individually bound to the bound first secondary antibodies 830, 850, and 870, respectively.

Accordingly, the amplified multi-fluorescence signal 800 according to another embodiment can be fabricated. That is, the multiple amplified fluorescence signals 801, 802, and 803 in which each of the different first secondary antibodies 830, 850, and 870 and each of the different second secondary antibodies 840, 860, and 880 are bound together while forming multi-antibody layers from each of the different primary antibodies 811, 812 and 813 can be simultaneously fabricated. Accordingly, the amplified multi-fluorescence signal 800 can be fabricated.

According to various embodiments, the amplified multi-fluorescence signal 800 may achieve a high image processing throughput. That is, the image processing throughput of the amplified multi-fluorescence signal 800 may be much higher than an image processing throughput of the fluorescence signal obtained from direct and indirect immunofluorescence. In order to confirm such a comparison, various embodiments were applied to an expansion microscope, that is, an super-resolution imaging technology. In this case, the multi-fluorescence signal 800 might have been amplified even after being expanded. In this case, DAPI dyeing was also applied to a mouse brain slice prior to the expansion. The multi-fluorescence signal 800 was fabricated by binding the secondary antibody pairs 821, 822, and 823 so that each of the secondary antibody pairs 821, 822, and 823 forms six layers from each of the primary antibodies 811, 812 and 813. As a result, the multi-fluorescence signal 800 was imaged at a high degree of signal amplification without a deformation of a protein structure after the expansion. Furthermore, a laser exposure time necessary to reach the same brightness in the same laser intensity can be reduced about five times in the multi-fluorescence signal 800 in which the secondary antibody pairs 821, 822, and 823 are composed of the six layers, compared to the multi-fluorescence signal 800 in which the secondary antibody pairs 821, 822, and 823 are composed of one layer. This enables the fluorescence signal to be amplified n times through n cyclic containing. Accordingly, this means that an imaging time can be reduced by 1/n times.

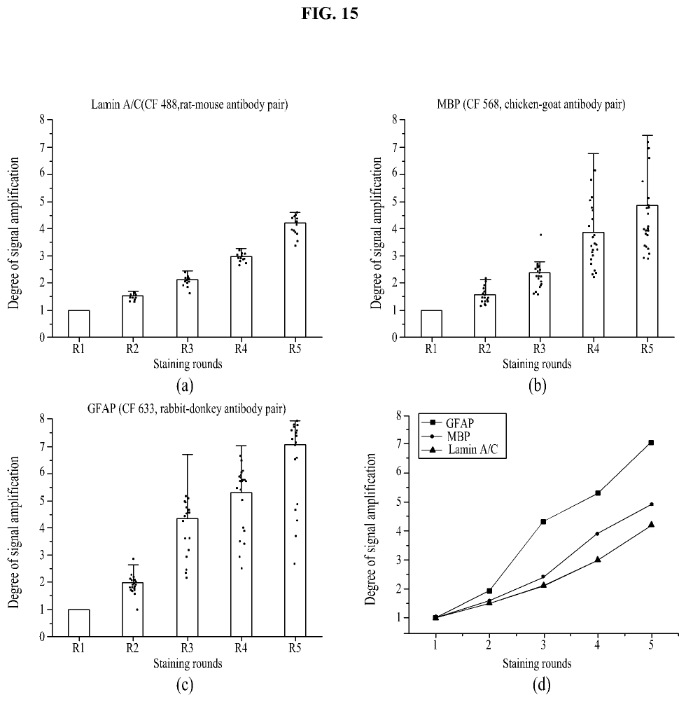

FIG. 15 is a diagram for describing a degree of signal amplification of the multi-fluorescence signal 800 according to various embodiments. In this case, FIGS. 15(a), 15(b) and 15(c) are bar graphs illustrating a degree of signal amplification of the fluorescence signals 801, 802, and 803 of the multi-fluorescence signal 800, respectively. FIG. 15(d) is a line graph for comparing the degree of signal amplification of the fluorescence signals 801, 802, and 803 of the multi-fluorescence signal 800.

Referring to FIG. 15, the multi-fluorescence signal 800 may be amplified. In this case, as the number of layers of each of the secondary antibody pairs 821, 822, and 823 of the respective primary antibodies 811, 812 and 813, that is, the staining rounds, is increased, a degree of signal amplification of each of the fluorescence signals 801, 802, and 803 may be increased. Accordingly, a degree of signal amplification of the multi-fluorescence signal 800 will be significantly increased.

Various embodiments provide a method of amplifying the multi-fluorescence signal 200 composed of multiple fluorescence signals 801, 802, and 803 amplified with respect to different types of target proteins p1, p2, and p3.

According to various embodiments, the method of amplifying the multi-fluorescence signal 800 may include a step (e.g., step 1310) of preparing different antibodies 830 and 840, 850 and 860, and 870 and 880 with which cross reactions have been removed, and a step (e.g., steps 1320, 1330, 1340, and 1350) of forming the fluorescence signals 801, 802, and 803 by binding the different antibody pairs 821, 822, and 823 based on combinations of the different antibodies 830 and 840, 850 and 860, and 870 and 880 to the different primary antibodies 811, 812 and 813 bound to the respective target proteins p1, p2, and p3, respectively.

According to various embodiments, the step (i.e., step 1310) of preparing the different antibodies 830 and 840, 850 and 860, and 870 and 880 may include a step of removing a cross reaction between each of the different antibodies 830 and 840, 850 and 860, and 870 and 880 and multiple orthogonal antibodies by using an agarose gel.

According to various embodiments, the different antibodies 830 and 840, 850 and 860, and 870 and 880 may have been conjugated with the fluorophores 831 and 841, 851 and 861, and 871 and 881, respectively.

According to various embodiments, the step of removing the cross reaction may be a step of removing a cross reaction while purifying each of the different antibodies 830 and 840, 850 and 860, and 870 and 880.

According to various embodiments, the step of removing the cross reaction may include a step (e.g., step 1411) of preparing the antibodies 1200 to be purified and different orthogonal antibodies 1202, 1203, 1204, and 1205 orthogonal to the antibodies 1200 to be purified, a step (e.g., step 1413) of putting all of the different orthogonal antibodies 1202, 1203, 1204, and 1205 in the agarose gel 1208, wherein the different orthogonal antibodies are connected to the agarose gel, a step (e.g., step 1415) of putting the antibodies 1200 to be purified in the agarose gel 1208, wherein some of the put antibodies 1200 are bound to the different orthogonal antibodies 1202, 1203, 1204, and 1205 through cross reactions, and a step (e.g., step 1417) of obtaining the remainder of the put antibodies 1200 as one of the different antibodies 830 and 840, 850 and 860, and 870 and 880.

According to various embodiments, the antibodies 1200 to be purified may have been conjugated with the fluorophores 1201.

According to various embodiments, the different orthogonal antibodies 1202, 1203, 1204, and 1205 may include all of orthogonal antibodies 1202, 1203, 1204, and 1205 orthogonal to the antibodies 1200 to be purified.

According to various embodiments, in the step (i.e., step 1413) of putting all of the different orthogonal antibodies 1202, 1203, 1204, and 1205 in the agarose gel 1208, an NHS group on a surface of the agarose gel 1208 and an amine group on a surface of each of the different orthogonal antibodies 1202, 1203, 1204, and 1205 react with each other to form an amide bond. Accordingly, the different orthogonal antibodies 1202, 1203, 1204, and 1205 may be connected to the agarose gel 1208.

According to various embodiments, the step of putting the antibodies 1200 to be purified in the agarose gel 1208 may be a step of removing the remaining NHS groups from the surface of the agarose gel 1208 and then putting the antibodies 1200 to be purified in the agarose gel 1208.

According to various embodiments, each of the different antibody pairs 821, 822, and 823 may be composed of at least one of the first secondary antibodies 830, 850, and 870 and at least one of the second secondary antibodies 840, 860, and 880 determined from two of the different antibodies 830 and 840, 850 and 860, and 870 and 880, respectively.

According to various embodiments, the step (i.e., steps 1320, 1330, and 1340) of forming the fluorescence signals 801, 802, and 803 may include a step of binding the different antibody pairs 821, 822, and 823 to the different primary antibodies 811, 812 and 813, respectively, in the form of multiple layers.

According to various embodiments, the step (i.e., steps 1320, 1330 and 1340) of forming the fluorescence signals 801, 802, and 803 may include a step (e.g., step 1320) of individually binding the different primary antibodies 811, 812 and 813 to the target proteins p1, p2, and p3, respectively, a step (e.g., step 1330) of individually binding, to the different primary antibodies 811, 812 and 813, the different first secondary antibodies 830, 850, and 870 determined from some of the different antibodies 830 and 840, 850 and 860, and 870 and 880, respectively, and a step (e.g., step 1340) of individually binding, to the different first secondary antibodies 830, 850, and 870, the different second secondary antibodies 840, 860, and 880 respectively determined from the remainder of the different antibodies 830 and 840, 850 and 860, and 870 and 880.

According to various embodiments, the step (i.e., steps 1320, 1330, and 1340) of forming the fluorescence signals 801, 802, and 803 may further include a step (e.g., step 1350) of binding the different first secondary antibodies 830, 850, and 870 to the different second secondary antibodies 840, 860, and 880, respectively, by individually adding the different first secondary antibodies 830, 850, and 870.

According to various embodiments, at least one of the step (i.e., step 1340) of individually binding the different second secondary antibodies 840, 860, and 880 or the step (i.e., step 1350) of binding the different first secondary antibodies 830, 850, and 870 by individually adding the different first secondary antibodies 830, 850, and 870 may be repeatedly performed.

According to various embodiments, the different primary antibodies 811, 812 and 813, the different antibody pairs 821, 822, and 823 or the fluorophores 831 and 841, 851 and 861, and 871 and 881 may be combined depending on the target proteins p1, p2, and p3 bound thereto, respectively.

According to various embodiments, a target species of each of the first secondary antibodies 830, 850, and 870 may be the same as a host species of each of the second secondary antibodies 840, 860, and 880. The host species of each of the first secondary antibodies 830, 850, and 870 may be the same as a target species of each of the second secondary antibodies 840, 860, and 880.

According to various embodiments, each of antibodies whose host species and target species are a rat and a mouse and antibodies whose host species and target species are a mouse and a rat, antibodies whose host species and target species are a chicken and a goat and antibodies whose host species and target species are a goat and a chicken, and antibodies whose host species and target species are a rabbit and a donkey and antibodies whose host species and target species are a donkey and a rabbit may be orthogonal to each other.

Various embodiments provide an image processing apparatus for processing an image through a multi-fluorescence signal amplified by the aforementioned method of amplifying the multi-fluorescence signal 800.

According to various embodiments, a fluorescence signal may be amplified. That is, various embodiments can simply implement an amplified fluorescence signal by using already commercialized antibodies and fluorophores. This enables the amplification and imaging of a fluorescence signal through a conventional fluorescence microscope without additional equipment. Furthermore, various embodiments enable a multi-fluorescence signal to be amplified using purified complementary antibody pairs. Furthermore, various embodiments can limit an additional problem in that a background signal within a clinical specimens may be increased because the purified complementary antibody pairs orthogonally act on biotin within the clinical specimens.

The present technology is different from signal amplification technologies that require specialized materials and additional modifications. In the case of signal amplification using gold nanoparticle complex, in order to generate and amplify a fluorescence signal, a fluorescent RNA probe having a fluorophore and a quencher connected to an end thereof, and a breakdown enzyme need to be used. An additional modification process of binding DNA to the gold nano particles is required. This corresponds to a method of generating decomposed and prohibited fluorescence without directly attaching a fluorescence signal substance to a mark, and is different from the present technology in which a composite body of an antibody to which fluorescence has been attached is expanded and a signal fixed to a mark is amplified. The present technology is a technology capable of amplifying a fluorescence signal without using DNA threads that are complicated and not commercialized a lot as in technologies, such as is HCR and bDNA.

The present technology is different from a tyramide signal amplification which requires a cyclic activation and deactivation process for multiple marker fluorescence imaging and has a single chemical mechanism. If a heat treatment process for deactivation is repeated, an antigenic determinant may be deformed, and a binding strength of antibodies may be decreased in a subsequent staining process. Furthermore, previously deposited tyramide may hinder a binding of an antibody for another mark in a subsequent staining and signal amplification step. However, the present technology can simultaneously perform multicolor fluorescence imaging by using orthogonal antibody pairs without additional activate and deactive processes.

The present technology is different from an avidin-biotin complex and labeled streptavidin-biotin signal amplification method having a single chemical mechanism. As described above, this technology has characteristics in that the amplification imaging of a multiple marker fluorescence signal is limited and a high background signal is a pathology sample containing a large amount of biotin. In contrast, the present technology enables the signal amplification of a multiple marker fluorescence signal by using secondary antibody pairs derived from several species and can be effectively used even in a pathology sample containing a large amount of biotin incidence.

The present technology is different from a DNA-based signal amplification technology that requires an optimization process for oligonucleotide-conjugated antibodies to label antigens for the purpose of immunostaining. The present technology does not require a custom optimization process because a secondary antibody conjugated with a fluorophore used in a conventional immunostaining method is used. This technology can be usefully even in typical biological.

Various embodiments may be applied to various fields as follows. Accordingly, effects in each field can be expected.

The first field is large-volume immunofluorescence imaging field having a high image processing throughput. Various tissue clearing techniques enable conventional two-dimension (2-D), to three-dimensional (3-D) large volumetric imaging. Such technologies will greatly improve a next-generation medical diagnostic system because they enable a microscopic structure of a cell and a tissue connectivity to be checked. Despite such an advantage, 3-D large volumetric imaging is very limited because a high scan time is required. However, such a problem can be solved by amplifying a fluorescence signal of a sample. The present technology will achieve the development of a 3-D large volumetric imaging having a high image processing throughput through signal amplification of immunofluorescence. That is, the present technology may be fabricated as a device, which amplifies a fluorescent intensity of a sample itself by substituting an immunostaining process after tissue clearing process in an already commercialized tissue clearing device with the present technology and has a high throughput capable of reducing a total imaging time.

The second field is pathologic analysis and diagnosis based on technology digital pathology and artificial intelligence. The digital pathology is one of technologies expected as a next-generation diagnostic system, and is a system for imaging clinical specimens and then storing the image in the form of a digital file, thereby enabling analysis and diagnosis. Digital pathology system is overcoming current limitations in pathology diagnostic system with a big data and artificial intelligence-based analytic system. The present technology, that provides a 3-D large volumetric imaging can be used along with conventional diagnostic technology. They can generate a high image processing throughput, which is necessary for digital pathology. Recently, in domestic and foreign markets, whole slide imaging and digital pathology for extracting and digitizing precise information by optically scanning whole pathology slides is actively developed. The present technology will provide a large amount of image data within the same time by being applied to the digital pathology. Furthermore, the present technology may be used along with various tissue clearing technologies, such as CLARITY, MAP, 3DISCO, CUBIC, and SHIELD, and will rapidly provide a 3-D large volumetric image.

The third field is a n vitro diagnostic multivariate index assay field. Various proteome and genome within the human body are used as indices on which a disease can be predicted based on the spatiotemporal distribution. A complicated system of a living body operates through an interaction between indices thereof in addition to the role of each of independent indices. Accordingly, simultaneous analysis of multiple biomarkers is important in vitro diagnostic multivariate in a for simultaneously analyzing multiple bio markers is very important. The present technology will provide an in diagnostic multivariate index assay by effectively amplifying a fluorescence signal of proteome and genome that develop at a low ratio within the human body. Because it can simultaneously amplify fluorescence signals having several types of biomarkers, and will provide an effective analytic system for drug response prediction.

The fourth field is a drug and medical device development. A clinical diagnostic field for predicting results of a treatment response and screening an effect of a biologic drug is continuously studied. with artificial intelligence-based technology development to provide an optimized disease prediction and prevention system. The present technology may provide a precise artificial intelligence-based analysis system through a complex interaction between pathological data and an advanced technology. A large amount of data processing by the present technology will provide sufficiently reinforced learning to the artificial intelligence-based analysis system. Accordingly, the advanced technology will help rational diagnosis and provide preventive services.

Various embodiments of this document and the terms used in the embodiments are not intended to limit the technology described in this document to a specific embodiment, but should be construed as including various changes, equivalents and/or alternatives of a corresponding embodiment. Regarding the description of the drawings, similar reference numerals may be used in similar elements. An expression of the singular number may include an expression of the plural number unless clearly defined otherwise in the context. In this document, an expression, such as “A or B”, “at least one of A and/or B”, “A, B or C” or “at least one of A, B and/or C”, may include all of possible combinations of listed items together. Expressions, such as “a first,” “a second,” “the first” or “the second”, may modify corresponding elements regardless of its sequence or importance, and are used to only distinguish one element from the other element and do not limit corresponding elements. When it is described that one (e.g., a first) element is “(functionally or communicatively) connected to” or “coupled with” the other (e.g., a second) element, one element may be directly connected to the other element or may be connected to the other element through another element (e.g., a third element).

The term “module” used in this document includes a unit configured as hardware, software or firmware, and may be interchangeably used with a term, such as logic, a logical block, a part or a circuit. The module may be an integrated part, a minimum unit to perform one or more functions, or a part thereof. For example, the module may be configured as an application-specific integrated circuit (ASIC).

According to various embodiments, each (e.g., a module or program) of the described elements may include a single entity or a plurality of entities. According to various embodiments, one or more elements or steps of the aforementioned elements may be omitted or one or more other elements or steps may be added. Alternatively or additionally, a plurality of elements (e.g., modules or programs) may be integrated into a single element. In such a case, the integrated element may perform a function performed by a corresponding one of the plurality of elements before at least one function of each of the plurality of elements is integrated identically or similarly. According to various embodiments, steps performed by a module, a program or another element may be executed sequentially, in parallel, repeatedly or heuristically, or one or more of the steps may be executed in different order or may be omitted, or one or more other steps may be added.

The embodiments of the invention in which an exclusive property or privilege is claimed are defined as follows:

1. A method of amplifying a fluorescence signal, comprising:
preparing a primary antibody and antibody pairs with respect to each target protein;
binding the primary antibody to the target protein; and
binding the antibody pairs to the primary antibody.

2. The method of claim 1, wherein the antibody pairs comprise first secondary antibodies conjugated with respective fluorophores and second secondary antibodies, and
wherein the binding of the antibody pairs to the primary antibody comprises:
binding at least one first secondary antibody to the primary antibody; and
binding at least one second secondary antibody to the first second antibody.

3. The method of claim 2, wherein the binding of the antibody pairs to the primary antibody further comprises binding at least one first secondary antibody to the second secondary antibody.
wherein the binding of the first secondary antibody and the binding of the second secondary antibody are repeatedly performed.

4. The method of claim 2, wherein the second secondary antibodies are conjugated with the fluorophores, respectively.

5. The method of claim 2, wherein:
a target species of the first secondary antibodies is identical with a host species of the second secondary antibodies, and
a host species of the first secondary antibodies is identical with a target species of the second secondary antibodies.

6. An image processing apparatus for processing an image by using a fluorescence signal amplified by the method of claim 1.

7. A method of amplifying multicolor fluorescence signals comprising multiple fluorescence signals amplified with respect to different types of target proteins, respectively, the method comprising:
preparing different antibodies for each of which a cross reaction has been removed; and
forming the fluorescence signals by binding different antibody pairs according to combinations of different antibodies to different primary antibodies bound to the respective target proteins, respectively,
wherein the preparing of the different antibodies comprises removing the cross reactions with multiple orthogonal antibodies with respect to each of the different antibodies by using an agarose gel.

8. The method of claim 7, wherein the different antibodies are conjugated with respective fluorophores.

9. The method of claim 7, wherein the removing of the cross reactions comprises removing the cross reactions while purifying each of the different antibodies.

10. The method of claim 7, wherein the removing of the cross reactions comprises:
preparing antibodies to be purified and different orthogonal antibodies orthogonal to the antibodies to be purified, respectively;
putting all of the different orthogonal antibodies in the agarose gel, wherein the different orthogonal antibodies are bound to the agarose gel;
putting the antibodies to be purified in the agarose gel, wherein some of the put antibodies are bound to the different orthogonal antibodies through cross reactions; and
obtaining a remainder of the put antibodies as one of the different antibodies,
wherein the antibodies to be purified are conjugated with fluorophores.

11. The method of claim 10, wherein the different orthogonal antibodies comprise all of orthogonal antibodies orthogonal to the antibodies to be purified, respectively.

12. The method of claim 10, wherein in the putting all of the different orthogonal antibodies in the agarose gel, as an NHS group on a surface of the agarose gel and an amine group on a surface of the different orthogonal antibodies react with each other to form an amide bond, the different orthogonal antibodies are connected to the agarose gel, and
wherein the putting of the antibodies to be purified in the agarose gel comprises putting the antibodies to be purified in the agarose gel after removing remaining NHS groups from the surface of the agarose gel.

13. The method of claim 7, wherein each of the different antibody pairs comprises at least one first secondary antibody and at least one second secondary antibody determined from two of the different antibodies, respectively.

14. The method of claim 7, wherein the forming of the fluorescence signals comprises binding the different antibody pairs to the different primary antibodies, respectively, in a form of multiple layers.

15. The method of claim 13, wherein the forming of the fluorescence signals comprises:
individually binding the different primary antibodies to the target proteins, respectively;
individually binding, to each of the different primary antibodies, different first secondary antibodies determined from some of the different antibodies; and
individually binding, to each of the different first secondary antibodies, different second secondary antibodies determined from a remainder of the different antibodies.

16. The method of claim 15, wherein the forming of the fluorescence signals further comprises binding the different first secondary antibodies to the different second secondary antibodies, respectively, by individually adding the different first secondary antibodies.

17. The method of claim 16, wherein at least one of the individually binding of the different second secondary antibodies or the binding of the different first secondary antibodies by individually adding the different first secondary antibodies is repeatedly performed.

18. The method of claim 8, wherein the different primary antibodies, the different antibody pairs or the fluorescence molecules are combined depending on a type of bound target protein.

19. The method of claim 13, wherein:
a target of the first secondary antibody is identical with a host of the second secondary antibody, and
a host of the first secondary antibodies is identical with a target of the second secondary antibody.

20. An image processing apparatus for processing an image by using multiple fluorescence signals amplified by the method of claim 7.
